(19) United States
(12) Patent Application Publication
(10) Pub. No.: US 2026/0337126 A1
(43) Pub. Date: Oct. 29, 2026

(54) HEAT-INTEGRATED METHOD FOR PRODUCING C2-C4 OLEFINS

(71) Applicant: BASF SE, Ludwigshafen am Rhein (DE)

(72) Inventors: Andre BADER, Ludwigshafen am Rhein (DE); Robert Peter Michael FRANZ, Ludwigshafen am Rhein (DE); Paul-Vinzent STROBEL, Ludwigshafen am Rhein (DE)

(21) Appl. No.: 19/110,165

(22) PCT Filed: Sep. 12, 2023
(86) PCT No.: PCT/EP2023/074996

§ 371 Date: Mar. 10, 2025

(30) Foreign Application Priority Data
Sep. 12, 2022 (EP) 22195146.0

(51) Int. Cl. C07C 1/24

(52) U.S. Cl. CPC C07C 1/24

(57) ABSTRACT

The invention relates to a method for producing C2-C4 olefins from dimethyl ether and optionally methanol, having the steps of: A) providing a dimethyl ether-containing flow A; B) mixing at least one part of the flow A with at least one hydrocarbon return flow R, which contains C2-C6 hydrocarbons, and with a water vapor flow G2, a feed flow B being obtained; C) heating the feed flow B to a temperature ranging from 430 to 500 °C in one or more heat exchangers and feeding same to an olefin fixed-bed reactor, wherein the heating process can also be carried out before individual sub-flows are mixed in order to form the feed flow B in step B); D) catalytically converting the feed flow B at a temperature ranging from 430 to 520 °C in order to form a product gas flow D, which contains C2-C4 olefins, additional C2-C6 hydrocarbons, methanol, and water vapor; E) cooling the product gas flow D to a temperature ranging from 170 to 220 °C in one or more heat exchangers by exchanging heat with the feed gas flow B; F) further cooling the product gas flow D to a temperature ranging from 35 to 65 °C by bringing the product gas flow into contact with at least one water-containing quenching circuit flow K, wherein water and methanol are condensed out of the product gas flow, and a hydrocarbon product gas flow F which is depleted of water and methanol is obtained; G) separating at least one sub-flow G1 of the at least one water-containing quenching circuit flow K and heating and evaporating the sub-flow G1 in one or more heat exchangers by exchanging heat with medium-pressure water vapor or heating and evaporating the sub-flow G1 by means of an electric heating process, a water vapor flow G2 being obtained, and feeding the water vapor flow G2 to step B); and H) separating one or more C2-C4 olefin-containing product flows P and obtaining at least one hydrocarbon return flow R, which contains C2-C6 hydrocarbons, from the hydrocarbon product gas flow F and returning same to step B).

The invention relates to a heat-integrated process for preparing C₂-C₄ olefins from dimethyl ether, with or without methanol.

It is known that propylene can be produced by converting a methanol/dimethyl ether mixture in a fixed bed reactor (methanol-to-propylene, MTP reactor). Known fixed bed reactors are operated with zeolite catalysts at temperatures of about 480 °C. For this purpose, it is necessary to heat up the methanol/DME-containing reactant gas stream to high temperatures prior to entry into the reactor.

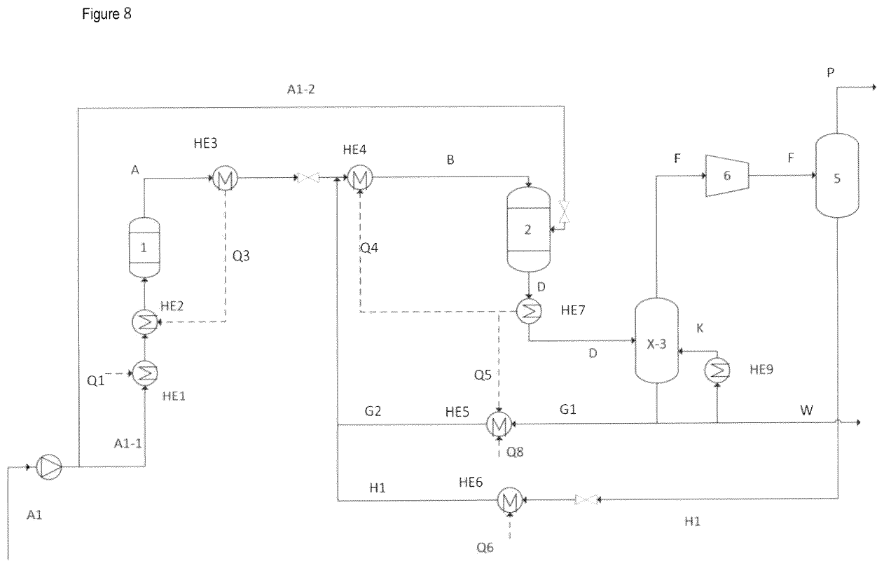

L. Jiang et al., Chinese Journal of Chemical Engineering 26 (2018), 2102-2111, describe a process for preparing propylene from methanol, in which methanol is first converted to dimethyl ether (DME) and then the product mixture is converted to propylene and further products. With reference to FIG. 8 of this publication, the process is described as follows: Methanol is preheated, evaporated and heated further by heat exchange with the product gas from the DME reactor in a heat exchanger. The methanol vapor is fed into the DME reactor at a temperature of 270 °C. A first substream of the product gas from the DME reactor is then cooled down to a temperature of 150 °C by heat exchange with a hydrocarbon recycle stream, cold methanol and circulating cooling water, and the gas/liquid mixture formed is separated in a phase separator. The gas phase is heated up again, while the liquid phase is cooled down further, and gas and liquid phase are fed into the individual trays of the propylene fixed bed reactor (MTP reactor). Alternatively, it is also possible to feed solely methanol into the individual trays of the MTP reactor. The second substream of the product gas from the DME reactor is mixed with the hydrocarbon recycle stream and water vapor, heated up to a temperature of about 460 °C with a burner and fed into the propylene reactor in which virtually all the methanol and DME are converted to propylene and further hydrocarbons.

A disadvantage of the process described is that a majority of the amount of heat required for heating of the reactant streams fed into the propylene reactor is provided by combustion of natural gas or other fossil hydrocarbons in a burner. This has a very adverse effect on the CO₂ balance of the overall process. Against the background of increasingly severe anthropogenic climate change, however, there is an increasing drive to implement industrial production processes having net emission of only a small amount of fossil-derived CO₂, or better none at all, into the atmosphere.

It is an object of the invention to provide a heat-integrated process for preparing C₂-C₄ olefins proceeding from dimethyl ether, in which it is possible to dispense with the provision of process heat by combustion of extrinsic fossil hydrocarbons in steady-state operation.

The object is achieved by a process for preparing C₂-C₄ olefins from dimethyl ether, with or without methanol, comprising the steps of:
A) providing a stream A comprising dimethyl ether;
B) mixing at least a portion of stream A with at least one hydrocarbon recycle stream R comprising C₂-C₆ hydrocarbons and a steam stream G2 to obtain a feed stream B;
C) heating feed stream B in one or more heat exchangers to a temperature in the range from 430 to 500 °C and feeding it into an olefin fixed bed reactor, where the heating may also precede the mixing of individual substreams to give feed stream B in step B);
D) catalytically converting feed stream B at a temperature in the range from 430 to 520 °C to a product gas stream D comprising C₂-C₄ olefins, further C₂-C₆ hydrocarbons, methanol and water vapor;
E) cooling product gas stream D in one or more heat exchangers to a temperature in the range from 170 to 220 °C by heat exchange with feed gas stream B;
F) further cooling product gas stream D to a temperature in the range from 35 to 65 °C by contacting with at least one water-comprising quench circulation stream K, with condensation of water and methanol, to obtain a water- and methanol-depleted hydrocarbon product gas stream F;
G) separating off at least one substream G1 of the at least one water-containing quench circulation stream K, and heating and evaporating substream G1 in one or more heat exchangers by heat exchange with mid-pressure steam, or heating and evaporating substream G1 by electrical heating, to obtain a steam stream G2, and feeding steam stream G2 in step B);
H) separating off one or more product streams P comprising C₂-C₄ olefins and obtaining at least one hydrocarbon recycle stream R comprising C₂-C₆ hydrocarbons from hydrocarbon product gas stream F and recycling into step B).

In one embodiment of the process of the invention, in step A), a stream A consisting essentially of DME (>90% by weight of DME) is fed into the process and divided into two substreams. The first substream A-1 comprises not more than 80% by weight of stream A and is used as described in step B). The second substream is mixed with a substream G3 of the water-containing quench circulation stream mentioned in step G) and fed as stream A-2 without further heating into one or more individual trays of the olefin fixed bed reactor in step C). It is also possible to operate the olefin fixed bed reactor isothermally. Isothermal operation can be effected, for example, in the manner described in WO2017/102096 A1. For instance, there may be heat transfer surfaces installed in the olefin fixed bed reactor that are operated, for example, with liquid salt or high-pressure steam as heat transfer medium. By virtue of the flow of the heat transfer medium through the heat transfer surfaces in the reactor, the heat of reaction that arises is removed from the reactor and hence this is operated isothermally.

In a preferred embodiment of the process of the invention, step A) comprises the feeding of a methanol-comprising feed gas stream A1 into a dimethyl ether fixed bed reactor and catalytic conversion of methanol to dimethyl ether to obtain a product gas stream comprising dimethyl ether, methanol and water vapor. If stream A1 comprises ethanol, product stream A will comprise not only dimethyl ether, methanol and water vapor but also ethanol and ethylene.

C₂-C₄ olefins are ethylene, propylene, 1-butene, 2-butenes and isobutene. Preference is given to preparing ethylene and/or propylene by the process of the invention.

In a preferred embodiment, the process of the invention thus comprises the steps of:
A) feeding a feed stream A1 comprising methanol into a dimethyl ether fixed bed reactor and catalytically converting methanol to dimethyl ether to obtain a product stream A comprising dimethyl ether, methanol and water vapor;
B) mixing at least a portion of product stream A with at least one hydrocarbon recycle stream R comprising C₂-C₆ hydrocarbons and a steam stream G2 to obtain a second feed stream B;
C) heating second feed stream B in one or more heat exchangers to a temperature in the range from 430 to 500 °C and feeding it into an olefin fixed bed reactor, where the heating may also precede the mixing of individual substreams to give feed stream B in step B);
D) catalytically converting feed stream B at a temperature in the range from 430 to 520 °C to a product gas stream D comprising ethylene, propylene, further C₂-C₆ hydrocarbons, methanol and water vapor;
E) cooling product gas stream D in one or more heat exchangers to a temperature in the range from 170 to 220 °C by heat exchange with feed stream B;
F) further cooling product gas stream D to a temperature in the range from 35 to 65 °C by contacting with at least one water-containing quench circulation stream K, with condensation of water and methanol, to obtain a water- and methanol-depleted hydrocarbon product gas stream F;
G) separating off at least one substream G1 of the at least one water-containing quench circulation stream K, and heating and evaporating substream G1 in one or more heat exchangers by heat exchange with mid-pressure steam, or heating and evaporating substream G1 by electrical heating, to obtain a steam stream G2, and feeding steam stream G2 in step B);
H) separating off one or more product streams P comprising ethylene and/or propylene and obtaining at least one hydrocarbon recycle stream R comprising C₂-C₆ hydrocarbons from hydrocarbon product gas stream F and recycling into step B).

In step A), preferably, a methanol-comprising feed stream A1 is fed into a dimethyl ether fixed bed reactor and methanol is catalytically converted to dimethyl ether to obtain a product stream A comprising dimethyl ether, methanol and water vapor.

A methanol-comprising feed stream A1, which preferably comes from an upstream methanol synthesis plant, is evaporated and heated up to a temperature of generally 250 to 300 °C, for example 275 °C, and fed into the dimethyl ether fixed bed reactor. The catalyst used is generally gamma-alumina. The conversion temperature is generally 250 to 400 °C; the pressure is generally 1 to 25 bar, for example 4 bar. The first product gas stream leaving the dimethyl ether fixed bed reactor is at a temperature of generally 350 to 400 °C, for example of 370 °C. The methanol conversion is generally 50% to 90%, preferably 65% to 85%, for example 75%. Product gas stream A is cooled by heat exchange with the methanol-comprising feed stream A1. The first product gas stream after heat exchange is at a temperature of generally 180 to 250 °C.

In a step B), at least a portion of this stream A is mixed with a hydrocarbon recycle stream comprising C₂-C₆ hydrocarbons and a steam stream G2 to obtain a feed gas stream B. In general, this portion of stream A is at least 50% by weight and preferably up to 80% by weight. A further portion A-2 of stream A of preferably at least 20% by weight can be fed directly to one or more trays of the olefin fixed bed reactor. If this portion of stream A comes from an upstream dimethyl ether reactor, it is generally cooled before being fed into the trays of the olefin fixed bed reactor, preferably to a temperature in the range from 30 to 60 °C. This substream A-2 is preferably fed into the reactor in liquid form.

The cooling by substream A-2 can also be dispensed with if the olefin fixed bed reactor is operated and cooled isothermally. Isothermal operation can be effected, for example, in the manner described in WO2017/102096 A1. For instance, there may be heat transfer surfaces installed in the olefin fixed bed reactor that are operated, for example, with liquid salt or high-pressure steam as heat transfer medium. By virtue of the flow of the heat transfer medium through the heat transfer surfaces in the reactor, the heat of reaction that arises is removed from the reactor and hence this is operated isothermally. In order to establish the desired product distribution in product gas stream D, it is also possible here to feed a substream A-2 to an intermediate stage of the olefin fixed bed reactor.

In a further embodiment, a methanol-comprising stream is fed directly to one or more trays of the olefin fixed bed reactor. Preference is given to feeding a portion of the methanol-comprising first feed stream A1 that comes, for example, from an upstream methanol synthesis plant directly into the olefin fixed bed reactor. This portion of the methanol-comprising feed stream A1 may be up to 60% by weight of the overall feed stream A1.

The hydrocarbon recycle stream R that comes from the removal of the C₂-C₄ olefins generally comprises C₄-C₆ hydrocarbons. Depending on whether ethylene, propylene or butenes are obtained as product of value in step H), the hydrocarbon recycle stream R may also comprise ethylene, propylene and/or butenes. If the amount of C₂-C₄ olefin obtained as product in step H) comprises propylene to an extent of at least 85% by weight, for example, stream R will consist of C₄-C₆ hydrocarbons to an extent of at least 50% by weight. The hydrocarbon recycle stream R prior to mixing is generally at a temperature in the range from 100 to 175 °C, preferably in the range from 130 to 160 °C. The hydrocarbon recycle stream R is preferably heated up by heat exchange with mid-pressure steam and is at a temperature prior to heating of generally 30 to 100 °C, preferably of 50 to 80 °C.

The product stream A from the dimethyl ether fixed bed reactor is also mixed with a steam stream G2. The steam stream G2 is generally at a temperature in the range from 100 to 200 °C, preferably in the range from 100 to 150 °C. According to the invention, this steam stream is heated up by heat exchange with mid-pressure steam.

The feed stream B thus obtained generally comprises 15% to 50% by weight, preferably 20% to 40% by weight, of water vapor. It generally further comprises 5% to 10% by weight of methanol, 10% to 20% by weight of dimethyl ether and 25% to 50% by weight of C₂-C₆ hydrocarbons.

In a step C), feed stream B is heated up in one or more heat exchangers to a temperature in the range from 430 to 500 °C and fed into an olefin fixed bed reactor. The heating may also precede the mixing of individual substreams in step B).

In general, feed stream B on feeding into the olefin fixed bed reactor is at a temperature in the range from 430 to 500 °C, for example 470 °C. Feed stream B is heated to that temperature by heat exchange with the (second) product gas stream D from the olefin fixed bed reactor. The inventive heating of steam stream G1 by heat exchange with mid-pressure steam makes it possible to attain a temperature of 430 to 500 °C in feed stream B by heat exchange with the (second) product gas stream D without requiring additional heating with a burner in steady-state operation. The small temperature differences between the two gas streams B and D increase the required heat transfer area. In plants having high production capacity, this can lead to disproportionately large heat exchangers. In such cases, the last part of the heating of feed stream B can also be conducted with an electrical heat exchanger, which increases the temperature difference in the heat exchangers between streams B and D. This reduces the requisite heat transfer area to an implementable degree. As long as the electrical power is being generated without combustion of fossil energy carriers, stream B is still heated without CO₂ emissions.

In one embodiment of the process of the invention, the second feed gas stream B is heated in step C) partly by means of an electrical heat exchanger.

This is followed in step D) by catalytic conversion in the olefin fixed bed reactor to a product gas stream D comprising ethylene, propylene, further C₂-C₆hydrocarbons, methanol and water vapor. The conversion is generally effected over a zeolite catalyst, preferably over a catalyst based on a ZSM-5 zeolite. The reaction temperature is generally 430 to 500 °C, preferably 460 to 480 °C. The pressure is generally is 1.3 to 4 bar. The resultant second product gas stream D is preferably of the following composition: 1% to 15% by weight of ethylene, 1% to 15% by weight of propylene, 35% to 70% by weight of water, 20% to 65% by weight of C₂-C₆ hydrocarbons, and also C₆⁺ hydrocarbons and 0.1% to 1.5% by weight of methanol and DME.

The olefin fixed bed reactor generally takes the form of a tray reactor. The number of trays is preferably 4 to 6. In one embodiment, a total of up to 50% by weight of gas stream A is fed directly to one or more trays of the olefin fixed bed reactor, preferably all trays of the olefin fixed bed reactor. In a further embodiment, a methanol-comprising stream is fed directly to one or more trays of the olefin fixed bed reactor, preferably all trays of the olefin fixed bed reactor.

The (second) product gas stream D on exit from the reactor is at a temperature of generally 430 to 520 °C, preferably 460 to 480 °C.

In a step E), product gas stream D is cooled in one or more heat exchangers to a temperature in the range from 160 to 220 °C by heat exchange with feed gas stream B. After this cooling step, the temperature of the (second) product gas stream D is generally 160 to 220 °C, preferably 170 to 210 °C, for example 190 °C.

In a step F), product gas stream D is cooled further to a temperature in the range from 35 to 60 °C by contacting with one or more water-containing quench circulation streams, with condensation of water and methanol, to obtain a water- and methanol-depleted hydrocarbon product gas stream F. The hydrocarbon product gas stream F thus obtained comprises essentially ethylene, propylene and further C₂-C₆ hydrocarbons.

In a step G), a portion of the water in the at least one quench circulation stream K is separated off, and this substream G1 is heated up and evaporated in accordance with the invention in one or more heat exchangers by heat exchange with mid-pressure steam, or this substream G1 is heated up and evaporated by electrical heating to obtain a heated steam stream G2. This heated steam stream G2 is mixed in step B) with the first product stream A (or feed stream A) originating from the dimethyl ether fixed bed reactor.

In general, the water-comprising quench circulation stream is heated up in step G) to an extent of at least 50%, based on the amount of heat supplied, by heat exchange with mid-pressure steam or via electrical heating. In addition, substream G1 of the water-containing quench circulation stream may additionally be heated and evaporated in step G) by heat exchange with product gas stream D.

Before being heated, the substream G1 separated off from quench circulation stream K is in liquid form and is generally at a temperature in the range from 70 to 100 °C. After being heated and evaporated, steam stream G1 is at a temperature in the range from 100 to 150 °C, preferably 120 to 140 °C, and correspondingly at a pressure of 1 to 6 bar, preferably of 2 to 4 bar.

In a first embodiment, the water stream G1 withdrawn from the quench circuit is heated by heat exchange with mid-pressure steam. Mid-pressure steam in the context of the present invention is steam at a temperature of at least 150 °C and a pressure of at least 5 bar, preferably at a temperature in the range from 150 to 250 °C, preferably 150 to 200 °C, and correspondingly a pressure in the range from 5 to 17 bar, preferably 5 to 11 bar.

All the stated pressures are absolute pressures.

The mid-pressure steam stream used in step G) preferably comes from a methanol synthesis upstream of step A) and/or from a synthesis gas preparation upstream of said methanol synthesis. The mid-pressure steam stream may come from spatially adjacent, separate production plants.

In a second embodiment, the water stream G1 withdrawn from the quench circuit is heated by electrical heating. Electrical heating can be effected, for example, in a tank with installed electrical heating elements.

According to the invention, the electrical energy supplied to the electrical heating is generated predominantly in a renewable manner, i.e. without combustion of fossil energy carriers. The electrical energy supplied to the electrical heating is thus provided largely without climatically harmful CO₂ emissions.

The mid-pressure steam may likewise be generated by electrical heating. Electrical heating can be effected, for example, in a tank with installed electrical heating elements.

In a step H), one or more product streams P comprising C₂-C₄ olefins are separated from hydrocarbon product gas stream F, and at least one recycle stream R comprising C₂-C₆ hydrocarbons is obtained. The overall recycle stream R is generally composed of multiple individual recycle streams. In general, the overall recycle stream R comprising C₂-C₆ hydrocarbons comprises essentially, i.e. to an extent of >95% by weight, C₂-C₆ hydrocarbons.

The hydrocarbon recycle stream R comprising C₂-C₆ hydrocarbons can likewise be heated up by heat exchange with mid-pressure steam.

The hydrocarbon recycle stream R comprising C₂-C₆ hydrocarbons may be heated up by heat exchange with product gas stream D prior to mixing with stream A to obtain feed stream B.

In general, step H) comprises steps H1) to H7):
H1) compressing hydrocarbon product gas stream F to obtain a liquid hydrocarbon stream H11 comprising propylene and C₄, C₅ and C₆⁺ hydrocarbons, and an ethane-, ethene- and propylene-comprising gaseous hydrocarbon stream H12;
H2) separating water from the liquid hydrocarbon stream H11 by phase separation to obtain a liquid hydrocarbon stream H21;
H3) separating a propylene-comprising stream H31 from the liquid hydrocarbon stream H21 to obtain a stream H32 comprising C₄, C₅ and C₆⁺ hydrocarbons; or separating a stream H31 comprising propylene and C₄ hydrocarbons to obtain a stream H32 comprising C₄, C₅ and C₆⁺ hydrocarbons;
H4) separating a stream H41 comprising C₆⁺ hydrocarbons from the stream H32 comprising C₄, C₅ and C₆⁺ hydrocarbons to obtain a stream H42 comprising C₄, C₅ and C₆ hydrocarbons; stream H41 optionally comprises aromatic C₆ hydrocarbons, and stream H42 aliphatic C₆ hydrocarbons;
H5) separating a propylene-comprising stream H51 from the ethane-, ethene- and propylene-comprising gaseous hydrocarbon stream H12 to obtain an ethane- and ethene-comprising stream H52;
H6) separating a stream H61 comprising butenes from the stream H42 comprising C₄, C₅ and C₆ hydrocarbons to obtain a stream H62 comprising C₅ and C₆ hydrocarbons; and/or separating a propylene-comprising stream H63 from stream H31 to obtain a stream H64 comprising butenes;
H7) obtaining at least one recycle stream R from one or more of the streams selected from stream H42 comprising C₄, C₅ and C₆ hydrocarbons, stream H62 comprising C₅ and C₆ hydrocarbons, the propylene-comprising stream H31, the propylene-comprising stream H51, the propylene-comprising stream H63, stream H61 comprising butenes, stream H64 comprising butenes, and the ethane- and ethene-comprising stream H52.

Steps H3), H4), H5) and H6) are conducted in standard distillation apparatuses. Useful distillation apparatuses in principle include the apparatuses known to the person skilled in the art for such separation tasks. As well as the actual column body with internals, the distillation column, as usual, also comprises a top condenser and a reboiler. The column body may have been equipped, for example, with structured packings, random packings or trays. The distillation apparatuses may be designed and operated by the common knowledge of the person skilled in the art.

The process of the invention for preparing C₂-C₄ olefins from dimethyl ether, with or without methanol, preferably follows on from a process for preparing methanol, wherein the process for preparing methanol comprises the following steps:
(a) producing a synthesis gas (II) comprising carbon monoxide, carbon dioxide and hydrogen from a hydrocarbonaceous feedstock (I) in a synthesis gas production unit;
(b) feeding the synthesis gas (II) from stage (a) to a methanol synthesis unit and converting it at a temperature of 150 to 300 °C and a pressure of 5 to 10 MPa abs in the presence of a methanol synthesis catalyst to a reaction mixture comprising methanol, water, carbon monoxide, carbon dioxide, hydrogen, dimethyl ether and methane, condensing a methanol- and water-enriched crude methanol stream (III) out of said reaction mixture, and conducting the crude methanol stream (III) and a gaseous stream (IV) comprising carbon monoxide, carbon dioxide, hydrogen and methane out of the methanol synthesis unit,
(c) expanding the crude methanol stream (III) from stage (b) in an expansion unit to a pressure of 0.1 to 2 MPa abs, and obtaining an expansion gas (V) comprising carbon dioxide and methane and a degassed crude methanol stream (VI) enriched with methanol and water;
(d) separating a carbon dioxide- and dimethyl ether-comprising low boiler stream (VII) by distillation from the degassed crude methanol stream (VI) from stage (c) in a distillation apparatus, and obtaining a methanol- and water-enriched bottom stream (VIII); and
(e) optionally separating a water-comprising high boiler stream (IX) from the bottom stream (VIII) from stage (d) in a further distillation apparatus, and obtaining methanol by distillation as stream (X). Step (e) can also be dispensed with since anhydrous methanol is not required.

The methanol- and water-enriched bottom stream (VIII) or, as the case may be, the pure methanol stream (X) may be used in the process of the invention.

The mid-pressure steam stream used in accordance with the invention in step G) of propylene preparation preferably comes from the synthesis gas production unit in step (a) of the methanol preparation and/or from the methanol synthesis unit in step (b) of the methanol preparation.

The process for preparing methanol preferably also comprises the following steps:
(f) feeding the carbon monoxide, carbon dioxide, dimethyl ether and methane components of value in streams (IV) and at least one of the two streams (V) and (VII) to a combustion unit and combusting them therein with supply of an exogenous gas (XI) having an oxygen content of 30% to 100% by volume, forming carbon dioxide-containing flue gas (XII);
(g) separating a carbon dioxide-enriched stream (XIV) from the carbon dioxide-containing flue gas (XII) from stage (f) in a carbon dioxide recovery unit to form an offgas stream (XIII); and
(h) recycling the carbon dioxide-enriched stream (XIV) separated off in the carbon dioxide recovery unit from stage (g) to the synthesis gas production unit of stage (a) and/or to the methanol synthesis unit of stage (b).

Such a process comprising steps (f), (g) and (h) is described in WO 2020/048809 A1.

The mid-pressure steam stream used in step G) of the preparation of C₂-C₄ olefins preferably comes from the synthesis gas production unit in step (a) of the methanol preparation and/or the methanol synthesis unit in step (b) of the methanol preparation.

Synthesis gas-producing feedstocks that may be used in this process are a wide variety of different carbonaceous feedstocks, regardless of whether they are in solid, liquid or gaseous form, and regardless of their chemical nature. For example, synthesis gas can be produced using either coal or hydrocarbons, and compounds comprising carbon and hydrogen.

Preferred carbonaceous feedstocks include natural gas, biogas, coal, wood, plastics, mineral oil, bionaphtha, or carbonaceous streams from mineral oil or natural gas processing, from chemical production methods, from renewable raw materials or from plastics recycling. In the case of coal or wood, synthesis gas is produced, for example, by a gasification process, also called coal gasification or wood gasification. Suitable feedstocks from mineral oil or natural gas processing are, for example, naphtha, LPG, gasoline, heavy oil or vacuum residue. What is meant by carbonaceous streams from chemical production processes is, for example, carbonaceous streams that are obtained as by-products and, rather than for purely thermal utilization, can also be used as feedstock for production of synthesis gas.

Particular preference is given to the use of methane-containing streams, and very particular preference to that of natural gas or biogas. It is possible here and even advantageous to feed carbon dioxide present in natural gas and in particular in biogas likewise to the synthesis gas production unit.

In the synthesis gas production unit, a synthesis gas (II) comprising carbon monoxide, carbon dioxide and hydrogen is first produced from the hydrocarbonaceous feedstock (I). Natural gas typically comprises 75% to 100% by volume of methane. Accompanying substances alongside methane include in particular the higher hydrocarbons ethane, propane, butane, but also ethene. Biogas typically comprises 40% to 75% by volume of methane and, as accompanying substances, essentially carbon dioxide, water, nitrogen and oxygen.

Typically, the synthesis gas (II) is produced by the production methods that are customarily used on an industrial scale, although the nature of the carbonaceous feedstock (I) also plays a role here. In the case of methane-containing feedstocks, for instance in the case of natural gas or biogas, the synthesis gas (II) is preferably produced in stage (a) by steam reforming, by autothermal reforming, by a combination of steam reforming and autothermal reforming, or by partial oxidation.

A particular advantage of partial oxidation is that the synthesis gas production unit need not be supplied with a separate fuel gas, and hence there is also no formation of a carbon dioxide-containing flue gas. In the partial oxidation, the energy required for synthesis gas production is obtained directly by partial oxidation from the methane-containing feedstock, and the combustion gases formed, carbon dioxide and carbon monoxide, are likewise used in the methanol synthesis. Therefore, preference is given to partial oxidation of methane-containing streams such as natural gas or biogas, for example.

The product stream from the partial oxidation has to be cooled. This is accomplished by means of heat exchangers with water as cooling medium. This evaporates and superheats the water, forming high-pressure steam (p=100-130 bar, T=300-450 °C). This high-pressure steam can be expanded to the required pressure and temperature level of mid-pressure steam in the range from 150 to 250 °C and 5 to 17 bar. Alternatively, removal of heat from the methanol synthesis reactor gives rise to heating steam in the pressure range from 5 to 17 bar.

The synthesis gas (II) produced comprises carbon monoxide, carbon dioxide and hydrogen, where the collective concentration thereof is typically 50% to 100% by volume, preferably ≥80% by volume and more preferably >90% by volume. Possible accompanying substances include, in particular, unconverted constituents of the carbonaceous feedstock used and by-products from the conversion thereof, for example nitrogen, argon, water or methane. Typically, the synthesis gas (II) comprises, as well as carbon monoxide, carbon dioxide and hydrogen, as a result of the preparation, also methane, and also nitrogen and argon, which has been introduced, for example, by the use of air in the synthesis gas production.

The conversion of the synthesis gas (II) is effected in a methanol synthesis unit at a temperature of 150 to 300 °C and a pressure of 5 to 10 MPa abs in the presence of a methanol synthesis catalyst. For this purpose, the synthesis gas (II) is typically compressed to the desired pressure by means of a compressor and converted in a reactor under the conditions specified.

The conversion is preferably effected at a temperature of ≥170 °C and more preferably at ≥190 °C, and preferably at ≤280 °C and more preferably at ≤260 °C. The conversion is effected preferably at >6 MPa abs and preferably at ≥9 MPa abs.

Reactors used may in principle be any reactors that are suitable for the exothermic conversion of synthesis gas to methanol under the process conditions specified. Reactors for synthesis of methanol from synthesis gas are common knowledge to the person skilled in the art. Examples of these include the adiabatic and quasi-isothermal reactors, variobar reactors and what are called double-wall superconverters that are mentioned in Ullmann's Encyclopedia of Industrial Chemistry, “Methanol” chapter, Section 5.2.1 “Reactor Design”, 2012 Wiley-VCH Verlag Gmbh & Co. KGaA, Weinheim, Germany.

Methanol synthesis catalysts are common knowledge to those skilled in the art. Examples include copper- and zinc-comprising heterogeneous catalysts. In general, these also comprise further elements, for example aluminum, rare earths or chromium.

The conversion of the synthesis gas (II) comprising carbon monoxide, carbon dioxide and hydrogen forms methanol and water.

A typical by-product formed is dimethyl ether. In addition, full hydrogenation of carbon monoxide or carbon dioxide also gives rise to methane as a further by-product. The reaction mixture produced in the reactor thus comprises methanol, water, dimethyl ether, carbon monoxide, carbon dioxide, hydrogen and methane. Additionally formed under the reaction conditions mentioned, however, are typically also further by-products, for example methyl formate, acetic acid, higher alcohols having carbon numbers of >2, esters and ethers having carbon numbers of >2, and also paraffins.

For separation of the complex reaction mixture, a methanol- and water-enriched crude methanol stream (III) is first condensed out. For this purpose, the reaction mixture produced in the reactor is typically fed to a condenser. Condensers used may be the apparatuses known to the person skilled in the art that are suitable for obtaining a methanol- and water-enriched condensate by controlled cooling under the present conditions. In general, the reaction mixture is cooled down to a temperature below the dew point of methanol. In accordance with the solubilities and vapor pressures of the components present in the reaction mixture, the methanol- and water-enriched crude methanol stream (III) still comprises gases dissolved therein, for example hydrogen, carbon monoxide, carbon dioxide, dimethyl ether, methane, and higher-boiling components than methanol. The crude methanol stream (III) condensed out is then discharged from the methanol synthesis unit for further workup and guided to stage (c).

The uncondensed gas stream especially comprises the unconverted carbon monoxide, carbon dioxide and hydrogen feedstocks, and methane. In order to obtain a high partial pressure of the synthesis gas components hydrogen, carbon monoxide and carbon dioxide, a portion of the uncondensed gas stream is typically discharged. If required, this discharged gas stream is sent to a removal of hydrogen in order to increase the partial pressure of hydrogen in the reactor. A higher partial pressure of hydrogen in the reactor reduces the formation of secondary components and especially also suppresses the Fischer-Tropsch reaction. The predominant portion of the uncondensed gas stream is recycled into the methanol synthesis unit as cycle gas and guided over the methanol synthesis catalyst in order to achieve maximum exploitation of the synthesis gas and hence high yields of methanol.

Accordingly, the methanol synthesis unit in stage (b) advantageously comprises a compressor for compression of the synthesis gas (II), a reactor for conversion of the synthesis gas (II), a condenser for condensing out the crude methanol stream (III), and a conduit for recycling of uncondensed gas to the reactor.

The uncondensed gas stream that has not been recycled as synthesis cycle gas is discharged from the methanol synthesis unit as gaseous stream (IV) and optionally guided to stage (f).

In stage (c) of the process, the crude methanol stream (III) that has been condensed out in stage (b) and discharged from the methanol synthesis unit is expanded in an expansion unit to a pressure of 0.1 to 2 MPa abs, and an expansion gas (V) comprising carbon dioxide and methane and a degassed crude methanol stream (VI) enriched with methanol and water are obtained. The expansion is typically effected in an apparatus in which gas phase and liquid phase can be efficiently separated from one another. Typically, the apparatus is a liquid separator. Suitable apparatuses for this purpose are known to those skilled in the art.

The expansion is preferably effected to a pressure of ≥0.2 MPa abs and more preferably to ≥0.4 MPa abs, and preferably to ≤1.5 MPa abs and more preferably to ≤1 MPa abs. In general, the temperature of the expanded mixture is 0 to 150 °C, preferably ≥10 °C and more preferably ≥20 °C, and preferably ≤120 °C and more preferably ≤60 °C.

The degassed crude methanol stream (VI) has been further enriched in methanol and water, but also comprises further components in accordance with the solubilities and vapor pressures of the components present in the crude methanol stream (III), for example gases dissolved therein such as hydrogen, carbon monoxide, carbon dioxide, dimethyl ether, methane or higher-boiling components than methanol.

The expansion gas (V) comprising carbon dioxide and methane is preferably guided to stage (f). Alternatively, however, the expansion gas (V) may also be discharged from the methanol synthesis plant and, for example, utilized thermally or disposed of in some other way. Preference is given, however, to utilization thereof within the methanol synthesis plant as feed stream to the combustion unit in stage (f).

In stage (d) of the process, the degassed crude methanol stream (VI) obtained in stage (c) is separated by distillation in a distillation apparatus into a low boiler stream (VII) comprising carbon dioxide and dimethyl ether, and a bottom stream (VIII) enriched with methanol and water.

The low boiler stream (VII) separated off by distillation comprises primarily carbon dioxide and dimethyl ether as low boilers that have been separated off and, based on the composition of the degassed crude methanol stream (VI), further low boilers, for example methane, and also, depending on the separation performance and mode of operation of the distillation apparatus, methanol or higher-boiling components than methanol, for example water. The low boiler stream (VII) comprising carbon dioxide and dimethyl ether is likewise preferably guided to stage (f). If the expansion gas (V) is guided to the combustion unit in stage (f) and hence reused directly within the methanol synthesis plant, the low boiler stream (VII) may alternatively also be discharged from the methanol synthesis plant and, for example, utilized thermally or disposed of in some other way. Preference is given, however, to utilization thereof within the methanol synthesis plant as feed stream to the combustion unit in stage (f).

The methanol- and water-enriched bottom stream (VIII) additionally also comprises further components that are higher-boiling than methanol, for example higher-boiling by-products than methanol from the methanol synthesis, for instance acetic acid, higher alcohols, higher esters, higher ethers or paraffins.

To obtain the methanol, finally, in stage (e), a water-containing high boiler stream (IX) is separated from the bottom stream (VIII) obtained in stage (d) in a further distillation apparatus and methanol is obtained by distillation as stream (X).

The high boiler stream (IX) comprises water and also further components that are higher-boiling than methanol, for example higher-boiling by-products than methanol from the methanol synthesis, for instance acetic acid, higher alcohols, higher esters, higher ethers or paraffins. This stream can be sent to a wastewater treatment, for example.

Via stream (X), methanol can be obtained in a high purity of ≥95% by weight, preferably ≥98% by weight and more preferably ≥99% by weight. Accompanying substances include residual amounts of low and high boilers that have not been removed completely by distillation, especially water, and very small amounts of ethanol, esters and ethers.

In the stepwise workup of the reaction mixture to obtain the methanol as stream (X), streams (IV), (V) and (VII) are separated off. However, these still contain components of value, for example carbon monoxide, carbon dioxide, methane and dimethyl ether. The aim is largely physical reuse of the carbon in these components of value for further synthesis of methanol with simultaneous avoidance of carbon dioxide emission. It is possible by the additional process steps (f), (g) and (h) to reuse the carbon in the components of value specifically for the further synthesis of methanol, i.e. to produce further product of value, and at the same time to avoid emission of carbon dioxide from the methanol synthesis. Steps (f), (g) and (h) are described in detail in WO 2020/048809 A1.

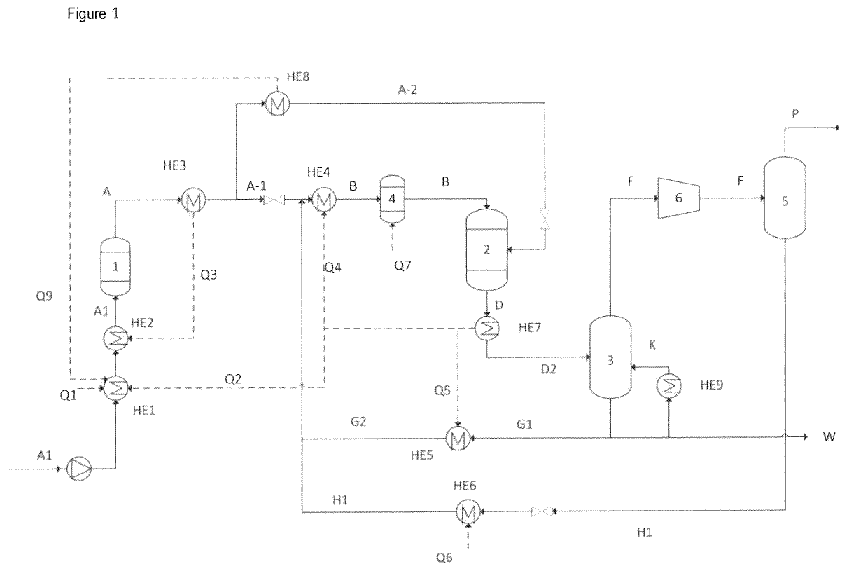

The invention is more particularly elucidated by FIGS. 1 to 10. The figures show:

FIG. 1: a noninventive procedure using a burner 4 for heating of feed stream B, with feeding of a DME-comprising substream A-2, after cooling in heat exchanger HE8, into individual trays of reactor 2 for cooling of reactor 2.

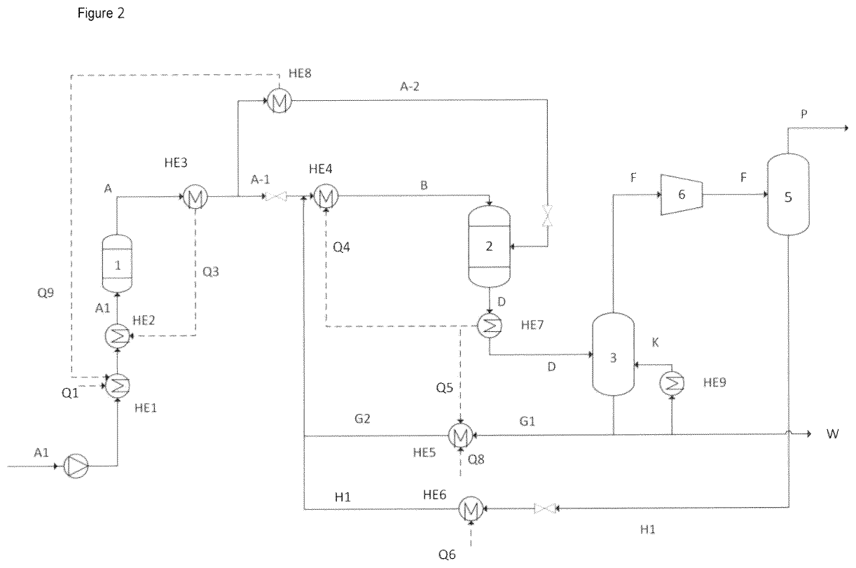

FIG. 2: an inventive procedure with feeding of a DME-comprising substream A-2, after cooling in heat exchanger HE8, into individual trays of olefin fixed bed reactor 2 for cooling of reactor 2.

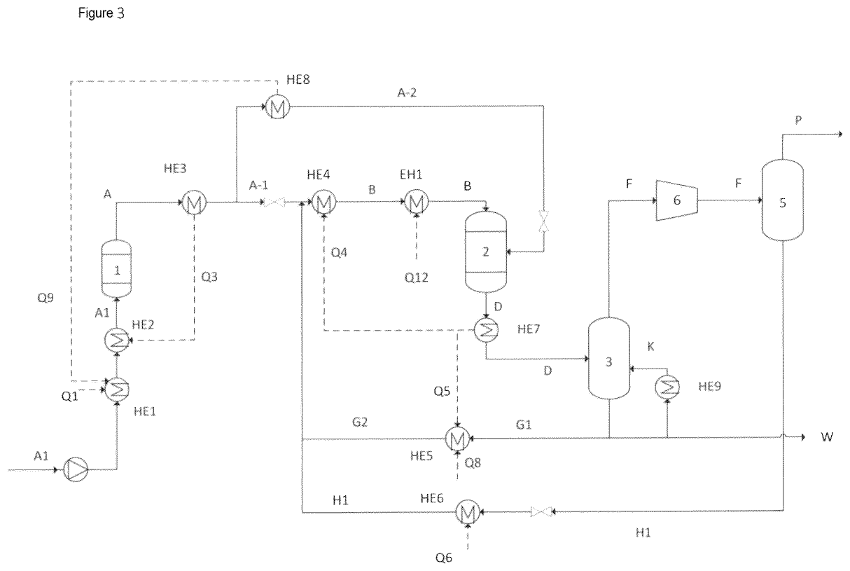

FIG. 3: an inventive procedure with feeding of a DME-comprising substream A-2, after cooling in heat exchanger HE8, into individual trays of olefin fixed bed reactor 2 for cooling of reactor 2. At the same time, electrical heat exchanger EH1 is used to increase the temperature difference in apparatus HE4 and hence to distinctly reduce the heat transfer area.

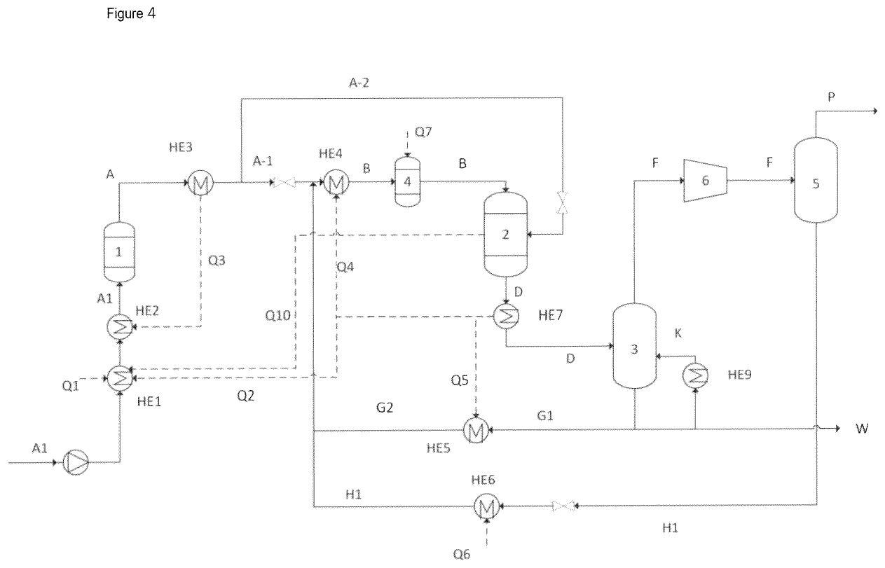

FIG. 4: a noninventive procedure using a burner 4 for heating of feed stream B, with feeding of a DME-comprising substream A-2, without cooling, into individual trays of olefin fixed bed reactor 2, and isothermal operation of reactor 2.

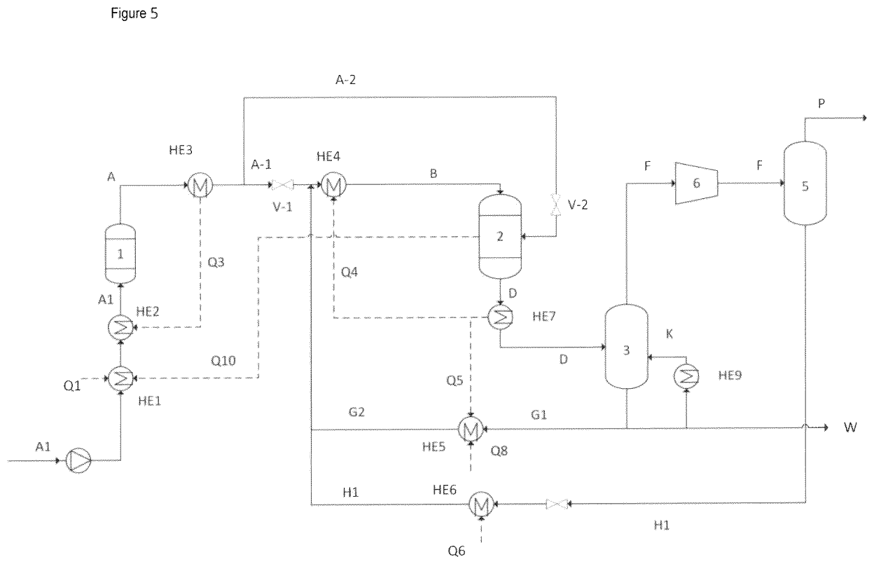

FIG. 5: an inventive procedure with feeding of a DME-comprising substream A-2, without cooling, into individual trays of olefin fixed bed reactor 2, and isothermal operation of reactor 2.

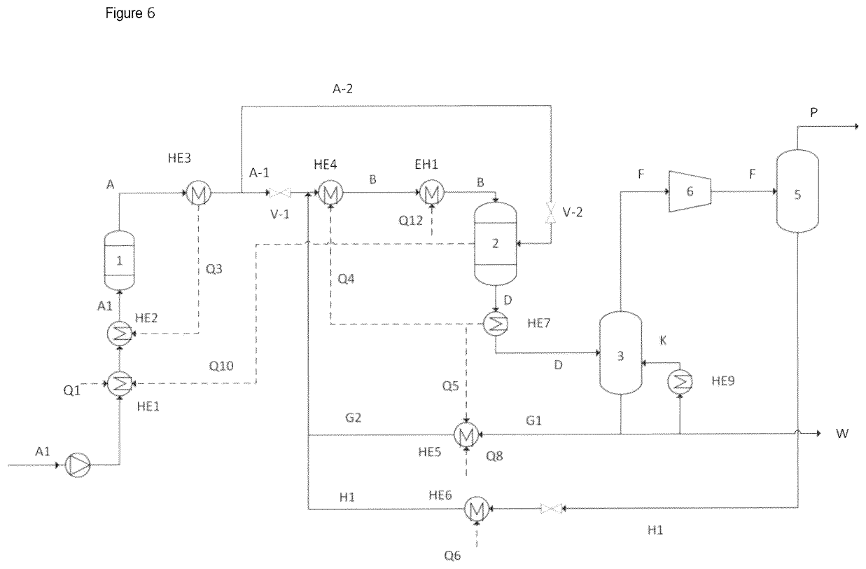

FIG. 6: an inventive procedure with feeding of a DME-comprising substream A-2, without cooling, into individual trays of olefin fixed bed reactor 2, and isothermal operation of reactor 2. At the same time, electrical heat exchanger EH1 is used to increase the temperature difference in apparatus HE4 and hence to distinctly reduce the heat transfer area.

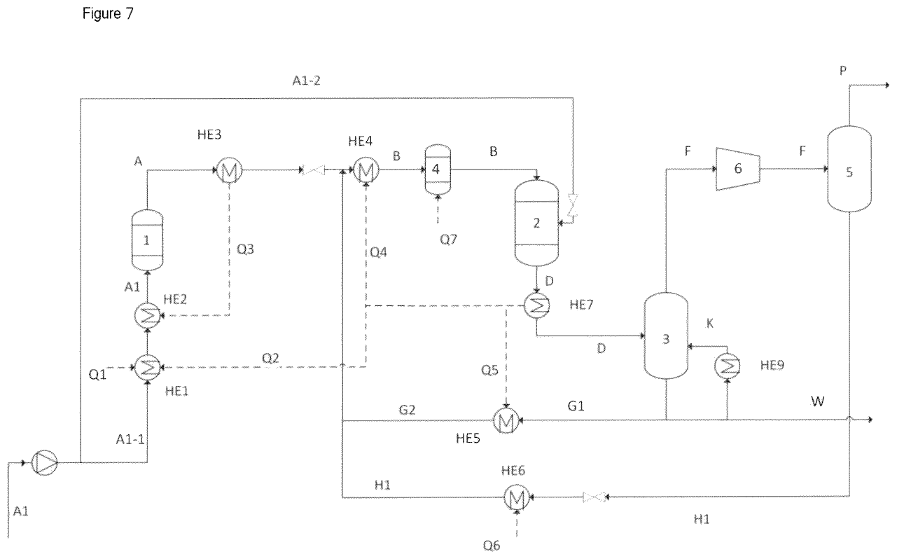

FIG. 7: a noninventive procedure using a burner 4 for heating of feed stream B, with feeding of a methanol substream A1-2 into individual trays of olefin fixed bed reactor 2 for cooling of reactor 2.

FIG. 8: an inventive procedure with feeding of a methanol substream A1-2 into individual trays of olefin fixed bed reactor 2 for cooling of reactor 2.

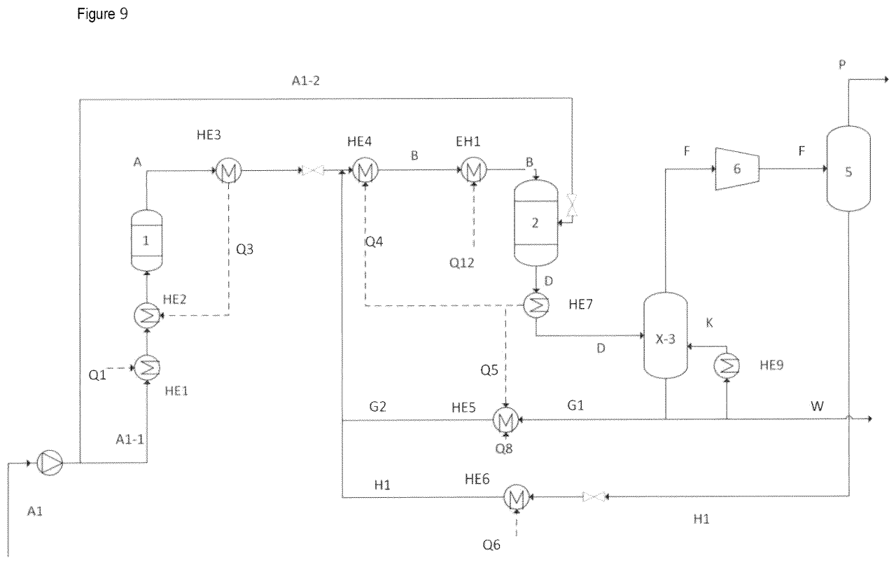

FIG. 9: an inventive procedure with feeding of a methanol substream A1-2 into individual trays of olefin fixed bed reactor 2 for cooling of reactor 2. At the same time, electrical heat exchanger EH1 is used to increase the temperature difference in apparatus HE4 and hence to distinctly reduce the heat transfer area.

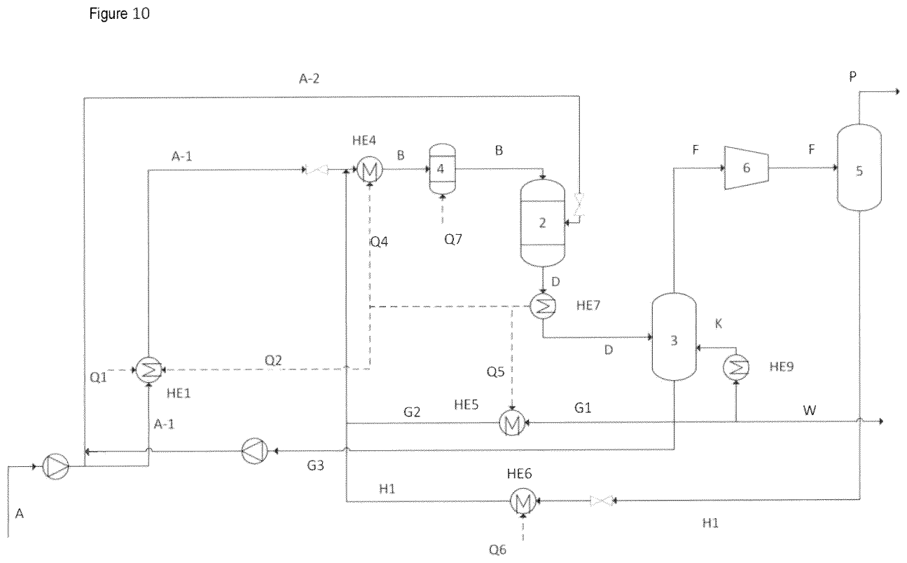

FIG. 10: a noninventive procedure without a DME prereactor, using a burner 4 for heating of feed stream B, with feeding of a DME substream A-2 into individual trays of olefin fixed bed reactor 2 for cooling of reactor 2.

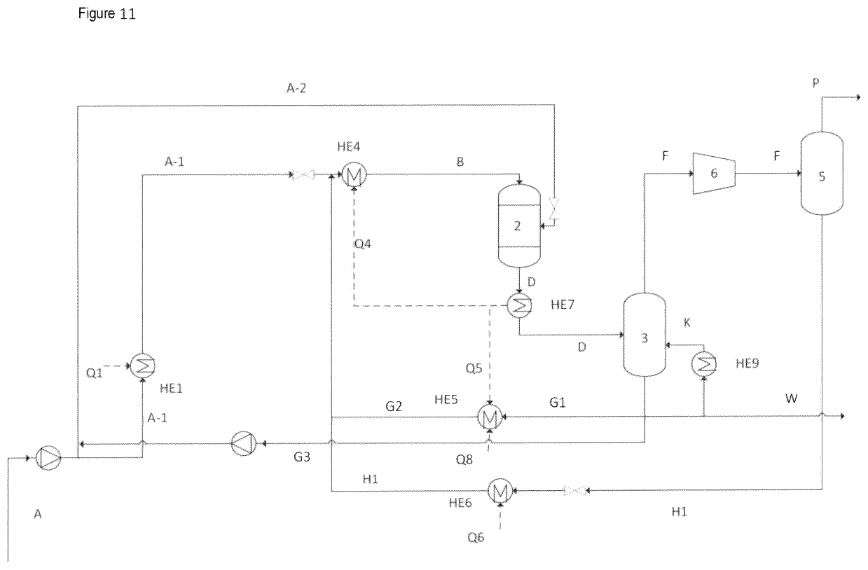

FIG. 11: an inventive procedure without a DME prereactor, with feeding of a DME substream A-2 into individual trays of olefin fixed bed reactor 2 for cooling of reactor 2.

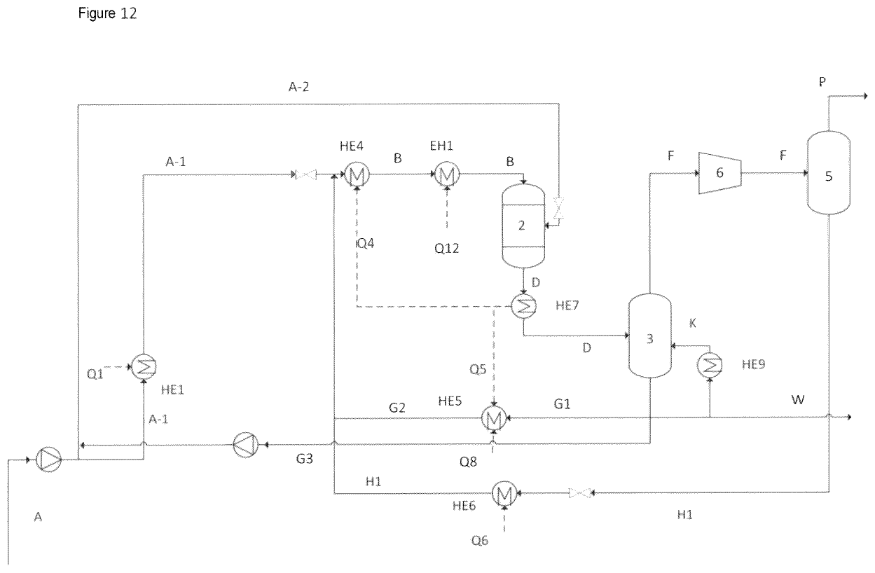

FIG. 12: an inventive procedure without a DME prereactor, with feeding of a DME substream A-2 into individual trays of olefin fixed bed reactor 2 for cooling of reactor 2. At the same time, electrical heat exchanger EH1 is used to increase the temperature difference in apparatus HE4 and hence to distinctly reduce the heat transfer area.

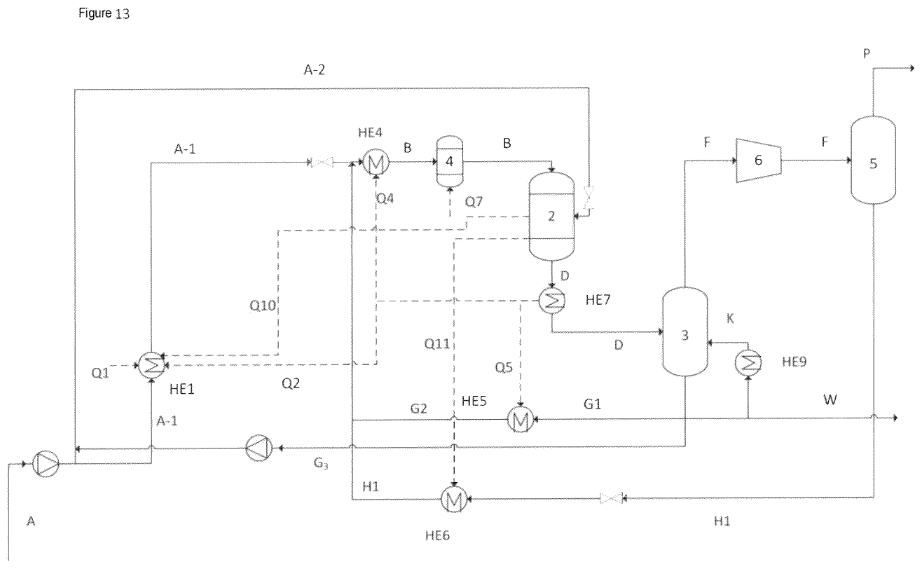

FIG. 13: a noninventive procedure without a DME prereactor, using a burner 4 for heating of feed stream B, with feeding of a DME substream A-2 into individual trays of olefin fixed bed reactor 2, and isothermal operation of reactor 2.

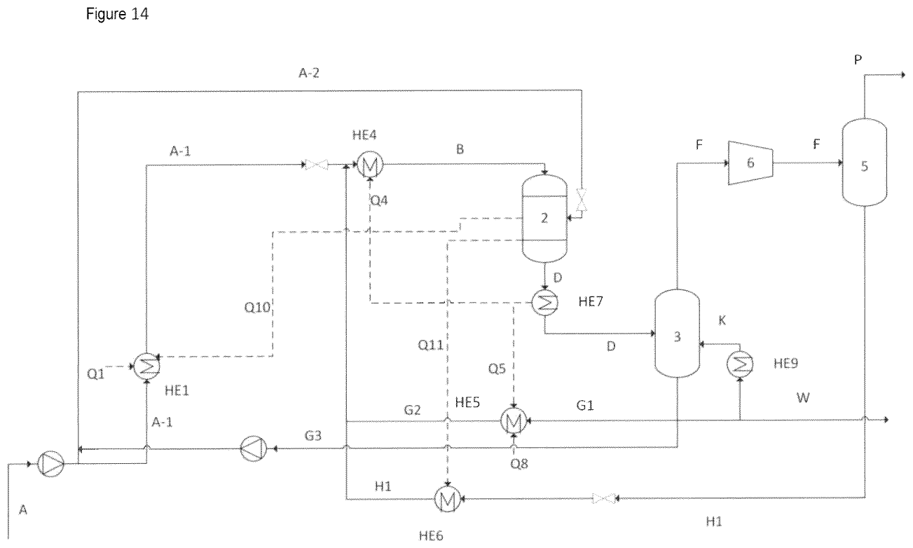

FIG. 14: an inventive procedure without a DME prereactor, with feeding of a DME substream A-2 into individual trays of olefin fixed bed reactor 2, and isothermal operation of reactor 2.

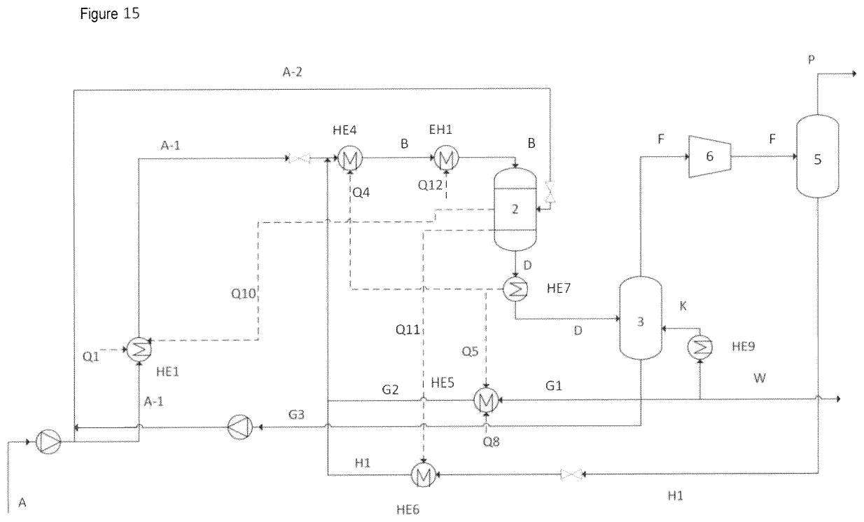

FIG. 15: an inventive procedure without a DME prereactor, with feeding of a DME substream A-2 into individual trays of olefin fixed bed reactor 2, and isothermal operation of reactor 2. At the same time, electrical heat exchanger EH1 is used to increase the temperature difference in apparatus HE4 and hence to distinctly reduce the heat transfer area.

### LIST OF REFERENCE SYMBOLS

1 DME prereactor
2 olefin fixed bed reactor
3 product gas quench
4 burner
5 product separation section
6 compressor
HE1 to HE9 heat exchangers
EH1 electrical heat exchanger
A1 methanol-comprising stream
A1-1, A1-2 substreams of A1
A DME-comprising stream
A-1, A-2 substreams of A
B feed stream comprising DME, recycled hydrocarbons and water vapor into the olefin fixed bed reactor
D product gas stream from the olefin fixed bed reactor
F product gas stream comprising hydrocarbons
G1, G2, G3 water streams
H1 hydrocarbon recycle stream
K quench water circulation stream
P product hydrocarbons
W wastewater stream
Q1 to Q12 heat flows

### Examples

Heat flows Q1 to Q11 as per the procedures according to FIGS. 1 to 15 were calculated by way of example for production of 60 t/h of propylene with minimal production of ethylene and butylene.

The results are collated in the table below.

| | FIG. | FIG. | FIG. | FIG. | FIG. | FIG. | FIG. | FIG. | FIG. | FIG. | FIG. | FIG. | FIG. | FIG. | FIG. |
|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 |
| Q₁ [MW] | 57 | 60 | 60 | 49 | 64 | 64 | 56 | 57 | 57 | 10 | 12 | 12 | 13 | 18 | 18 |
| Q₂ [MW] | 3 | — | — | 15 | — | — | 1 | — | — | 2 | — | — | 4 | — | — |
| Q₃ [MW] | 14 | 14 | 14 | 14 | 14 | 14 | 12 | 12 | 12 | — | — | — | — | — | — |
| Q₄ [MW] | 43 | 93 | 62 | 30 | 89 | 64 | 44 | 94 | 63 | 23 | 97 | 68 | 7 | 91 | 68 |
| Q₅ [MW] | 62 | 15 | 46 | 62 | 19 | 44 | 62 | 13 | 44 | 73 | 2 | 30 | 86 | 7 | 30 |
| Q₆ [MW] | 14 | 14 | 14 | 14 | 14 | 14 | 14 | 14 | 14 | 14 | 14 | 14 | — | — | — |
| Q₇ [MW] | 50 | — | — | 58 | — | — | 49 | — | — | 73 | — | — | 84 | — | — |
| Q₈ [MW] | — | 47 | 16 | — | 43 | 18 | — | 49 | 18 | — | 71 | 42 | — | 79 | 56 |
| Q₉ [MW] | 36 | 36 | 36 | — | — | — | — | — | — | — | — | — | — | — | — |
| Q₁₀ [MW] | — | — | — | 32 | 32 | 32 | — | — | — | — | — | — | 2 | 2 | 2 |
| Q₁₁ [MW] | — | — | — | — | — | — | — | — | — | — | — | — | 14 | 14 | 14 |
| Q₁₂ [MW] | — | — | 31 | — | — | 25 | — | — | 31 | — | — | 29 | — | — | 23 |

1.-13. (canceled)

14. A process for preparing C₂-C₄ olefins from dimethyl ether, with or without methanol, comprising the steps of:
A) providing a stream A comprising dimethyl ether;
B) mixing at least a portion of stream A with at least one hydrocarbon recycle stream R comprising C₂-C₆ hydrocarbons and a steam stream G2 to obtain a feed stream B;
C) heating feed stream B in one or more heat exchangers to a temperature in the range from 430 to 500 °C and feeding it into an olefin fixed bed reactor, where the heating may also precede the mixing of individual substreams to give feed stream B in step B);
D) catalytically converting feed stream B at a temperature in the range from 430 to 520 °C to a product gas stream D comprising C₂-C₄ olefins, further C₂-C₆ hydrocarbons, methanol and water vapor;
E) cooling product gas stream D in one or more heat exchangers to a temperature in the range from 170 to 220 °C by heat exchange with feed gas stream B;
F) cooling product gas stream D to a temperature in the range from 35 to 65 °C by contacting with at least one water-comprising quench circulation stream K, with condensation of water and methanol, to obtain a water- and methanol-depleted hydrocarbon product gas stream F;
G) separating off at least one substream G1 of the at least one water-containing quench circulation stream K, and heating and evaporating substream G1 in one or more heat exchangers by heat exchange with mid-pressure steam, or heating and evaporating substream G1 by electrical heating, to obtain a steam stream G2, and feeding steam stream G2 in step B); and
H) separating off one or more product streams P comprising C₂-C₄ olefins and obtaining at least one hydrocarbon recycle stream R comprising C₂-C₆ hydrocarbons from hydrocarbon product gas stream F and recycling into step B).

15. The process according to claim 14, wherein the second feed gas stream B is heated in step C) without using additional heat generated by combustion of fossil energy carriers.

16. The process according to claim 15, wherein the second feed gas stream B is heated in step C) partly by means of an electrical heat exchanger.

17. The process according to claim 14, wherein the mid-pressure steam stream is at a temperature in the range from 150 to 250 °C and a pressure in the range from 5 to 17 bar.

18. The process according to claim 14, wherein the mid-pressure steam stream is generated in a methanol synthesis upstream of step A) or in a synthesis gas preparation upstream of said methanol synthesis.

19. The process according to claim 14, wherein substream G1 of the water-containing quench circulation stream is additionally heated and evaporated in step G) by heat exchange with product gas stream D.

20. The process according to claim 14, wherein substream G1 of the water-containing quench circulation stream is heated in step G) to an extent of at least 50%, based on the amount of heat supplied, by heat exchange with mid-pressure steam.

21. The process according to claim 14, wherein the hydrocarbon recycle stream H1 comprising C₂-C₆ hydrocarbons is additionally heated up by heat exchange with mid-pressure steam.

22. The process according to claim 14, wherein step H) comprises:
H1) compressing hydrocarbon product gas stream F to obtain a liquid hydrocarbon stream H11 comprising propylene and C₄, C₅ and C₆⁺ hydrocarbons, and an ethane-, ethene- and propylene-comprising gaseous hydrocarbon stream H12;
H2) separating water from the liquid hydrocarbon stream H11 by phase separation to obtain a liquid hydrocarbon stream H21;
H3) separating a propylene-comprising stream H31 from the liquid hydrocarbon stream H21 to obtain a stream H32 comprising C₄, C₅ and C₆⁺ hydrocarbons; or separating a stream H31 comprising propylene and C₄ hydrocarbons to obtain a stream H32 comprising C₄, C₅ and C₆⁺ hydrocarbons;
H4) separating a stream H41 comprising C₆⁺ hydrocarbons from the stream H32 comprising C₄, C₅ and C₆⁺ hydrocarbons to obtain a stream H42 comprising C₄, C₅ and C₆ hydrocarbons; stream H41 optionally comprises aromatic C₆ hydrocarbons, and stream H42 aliphatic C₆ hydrocarbons;
H5) separating a propylene-comprising stream H51 from the ethane-, ethene- and propylene-comprising gaseous hydrocarbon stream H12 to obtain an ethane- and ethene-comprising stream H52;
H6) separating a stream H61 comprising butenes from the stream H42 comprising C₄, C₅ and C₆ hydrocarbons to obtain a stream H62 comprising C₅ and C₆ hydrocarbons; or separating a propylene-comprising stream H63 from stream H31 to obtain a stream H64 comprising butenes; and
H7) obtaining at least one recycle stream R from one or more of the streams selected from stream H42 comprising C₄, C₅ and C₆ hydrocarbons, stream H62 comprising C₅ and C₆ hydrocarbons, the propylene-comprising stream H31, the propylene-comprising stream H51, the propylene-comprising stream H63, stream H61 comprising butenes, stream H64 comprising butenes, and the ethane- and ethene-comprising stream H52.

23. The process according to claim 14, wherein step A) the feeding of a methanol-comprising feed stream A1 into a dimethyl ether fixed bed reactor and catalytic conversion of methanol to dimethyl ether to obtain a product stream A comprising dimethyl ether, methanol and water vapor.

24. The process according to claim 23, wherein the methanol fed in in step A) is produced in an upstream methanol preparation comprising the steps of:
(a) producing a synthesis gas (II) comprising carbon monoxide, carbon dioxide and hydrogen from a hydrocarbonaceous feedstock (I) in a synthesis gas production unit;
(b) feeding the synthesis gas (II) from stage (a) to a methanol synthesis unit and converting it at a temperature of 150 to 300 °C and a pressure of 5 to 10 MPa abs in the presence of a methanol synthesis catalyst to a reaction mixture comprising methanol, water, carbon monoxide, carbon dioxide, hydrogen, dimethyl ether and methane, condensing a methanol- and water-enriched crude methanol stream (III) out of said reaction mixture, and conducting the crude methanol stream (III) and a gaseous stream (IV) comprising carbon monoxide, carbon dioxide, hydrogen and methane out of the methanol synthesis unit;
(c) expanding the crude methanol stream (III) from stage (b) in an expansion unit to a pressure of 0.1 to 2 MPa abs, and obtaining an expansion gas (V) comprising carbon dioxide and methane and a degassed crude methanol stream (VI) enriched with methanol and water;
(d) separating a carbon dioxide- and dimethyl ether-comprising low boiler stream (VII) by distillation from the degassed crude methanol stream (VI) from stage (d) in a distillation apparatus, and obtaining a methanol- and water-enriched bottom stream (VIII); and
(e) optionally separating a water-comprising high boiler stream (IX) from the bottom stream (VIII) from stage (d) in a further distillation apparatus, and obtaining methanol by distillation as stream (X).

25. The process according to claim 24, wherein the methanol preparation also comprises steps (f) to (h):
(f) feeding the carbon monoxide, carbon dioxide, dimethyl ether and methane components of value in streams (IV) and at least one of the two streams (V) and (VII) to a combustion unit and combusting them therein with supply of an oxygenous gas (XI) having an oxygen content of 30% to 100% by volume, forming carbon dioxide-containing flue gas (XII);
(g) separating a carbon dioxide-enriched stream (XIV) from the carbon dioxide-containing flue gas (XII) from stage (f) in a carbon dioxide recovery unit to form an offgas stream (XIII); and
(h) recycling the carbon dioxide-enriched stream (XIV) separated off in the carbon dioxide recovery unit from stage (g) to the synthesis gas production unit of stage (a) and/or to the methanol synthesis unit of stage (b).

26. The process according to claim 24, wherein the mid-pressure steam stream used in step G) of preparation of C₂-C₄ olefins comes from the synthesis gas production unit in step (a) of the methanol preparation or from the methanol synthesis unit in step (b) of the methanol preparation.
